(19) United States
(12) Patent Application Publication
(10) Pub. No.: US 2010/0274348 A1
(43) Pub. Date: Oct. 28, 2010

(54) Modular Stent Assembly

(75) Inventors: Silvio Schaffner, Berlingen (CH); Thomas Bauer, Frauenfeld (CH)

Correspondence Address: HARNESS, DICKEY, & PIERCE, P.L.C, 7700 Bonhomme, Suite 400, ST. LOUIS, MO, 63105, US

(73) Assignee: Invatec Technology Center GmbH, Frauenfeld (CH)

(21) Appl. No.: 12/809,232

(22) PCT Filed: Dec. 19, 2007
(86) PCT No.: PCT/IB07/04007

§ 371 Date: Jun. 18, 2010

(51) Int. Cl. A61F 2/82

(52) U.S. Cl. 623/1.16

(57) ABSTRACT

The present invention relates to an assembly (10) comprising at least a first stent (1′) and a second stent (1′). Each stent comprises a proximal section (2), a central section (3), and a distal section (4). The proximal and distal sections provide a radial force which is essentially equal to a half of the radial force which is provided by the central section. Thus, by overlapping the distal section (4′) of the second stent (1′) to the proximal section (2′) of the first stent (1′), the radial force which is provided by the overlapped sections is nearly equal to the radial force which is provided by the central sections (3′, 3′) of the two stents.

The present invention relates to a modular stent assembly, i.e. an assembly of endoluminal prostheses, of which several examples can be serially implanted in a single blood vessel.

The use of stents is known to provide an inner support to the walls of blood vessels which tend to obstruct due to diseases and/or lesions such as stenosis. The stent, brought in a collapsed condition inside the vessel, adopts an expanded condition within the length affected by the stenosis. In order to provide a suitable support to the vessel walls, the stent has to exert a preset radial force which is outwardly directed. The extent of such radial force is one of the design criteria for the stents.

In some cases, the disease-affected blood vessel length is so high to overcome the individual stent length. In such case, it is possible to implant in series more stents of the known type, but this solution is not without drawbacks.

In fact, an individual stent is designed to be separately implanted. For this reason, the radial force which the stent must provide is determined during the design step on the basis of the vessel requirements.

The implant of several stents in series can be accomplished by juxtaposition or partial overlapping of adjacent stents.

In the case of a juxtaposition, the operation become extremely difficult, since it requires an accuracy which is generally not easy to be achieved. Therefore, this solution brings about the actual risk that a gap is left between the two adjacent stents, thus leaving a vessel length without any supports. Therefore, such vessel length is destined to a contraction, which is likely to decrease, again, the vessel section, thus hindering the surgery.

On the contrary, the partial overlapping of two adjacent stents gives origin, in the vessel length where the stents are overlapped, to a radial force which is twice the design radial force.

Thus, object of the present invention is to at least partially solve the problem set forth above with reference to the prior art.

Such problem is solved by a stent assembly in accordance with claim 1.

Further features and advantages of the present invention will be more clearly understood from the description of some exemplary embodiments, given herein below by way of non-limiting example, with reference to the following figures:

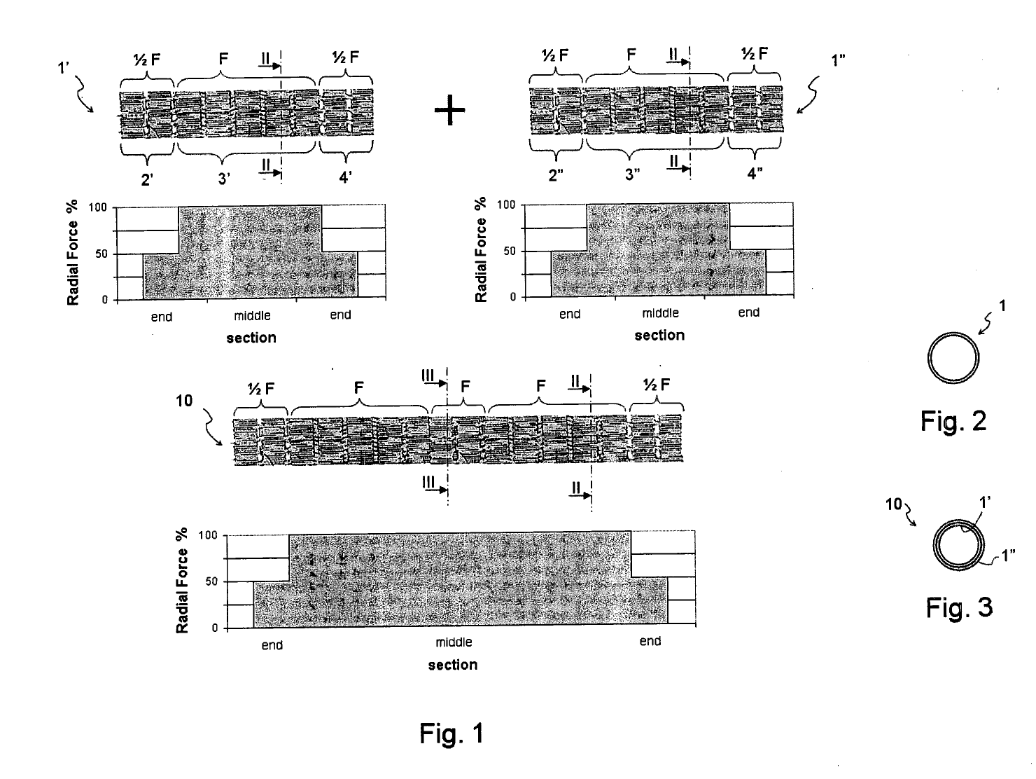

FIG. 1 schematically represents a stent assembly according to the invention, in a separated configuration and in an overlapped configuration, in which each configuration is accompanied by a respective radial force diagram;

FIG. 2 schematically represents a section along any line II-II in FIG. 1;

FIG. 3 schematically represents a section along the line in FIG. 1;

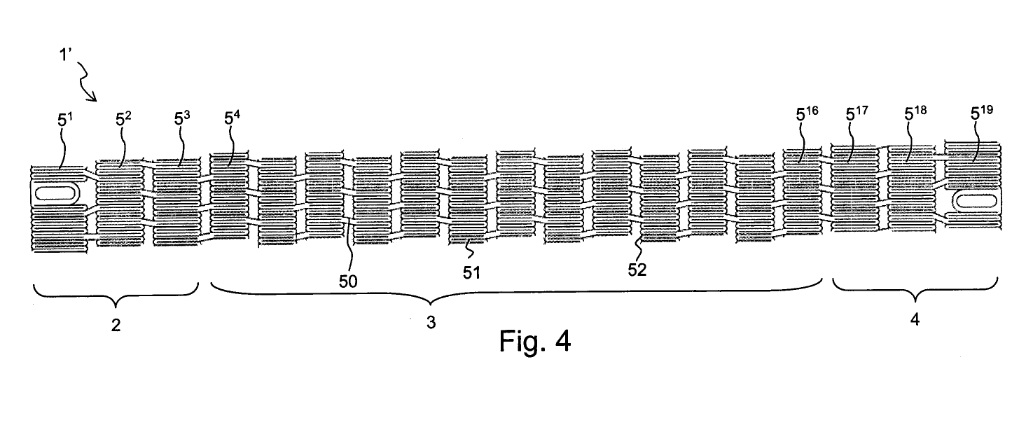

FIG. 4 represents the planar development of a stent according to the invention;

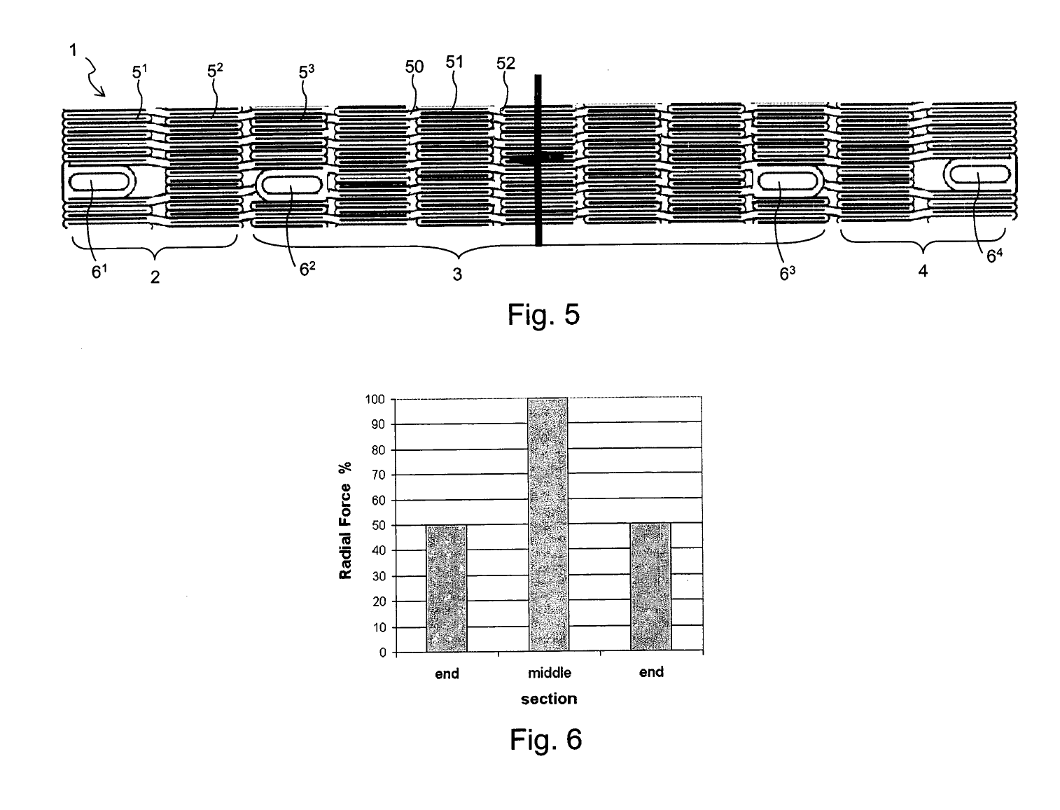

FIG. 5 represents the planar development of a stent according to the invention;

FIG. 6 represents a graphic of the radial forces provided by a stent according to the invention, at the ends and at the central section;

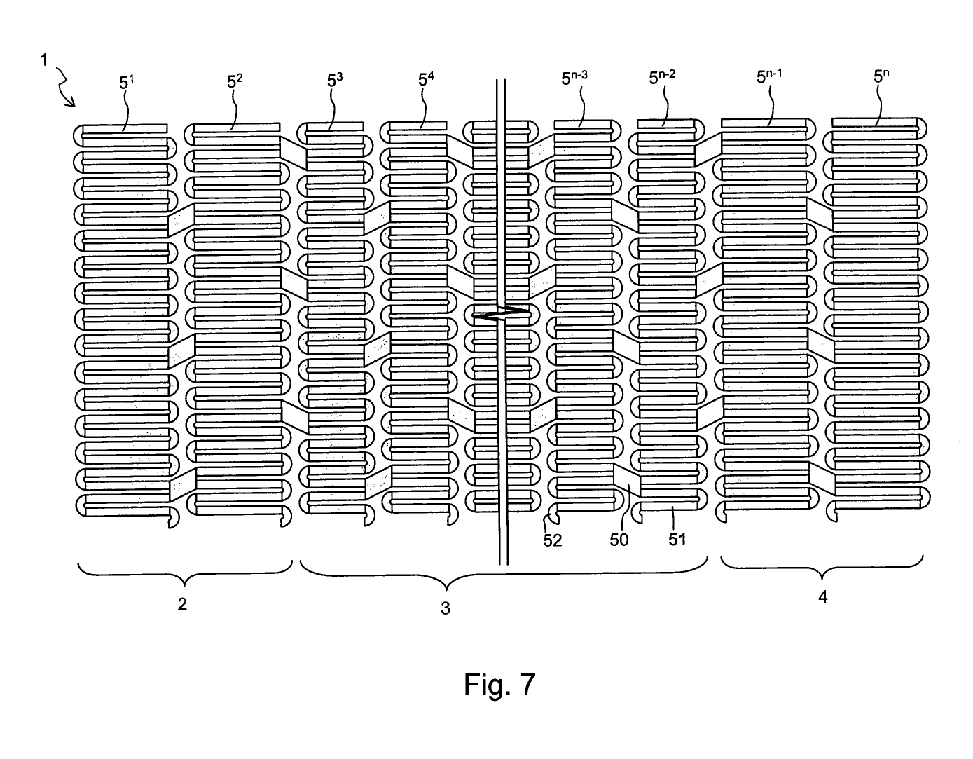

FIG. 7 schematically represents the planar development of a stent according to the invention;

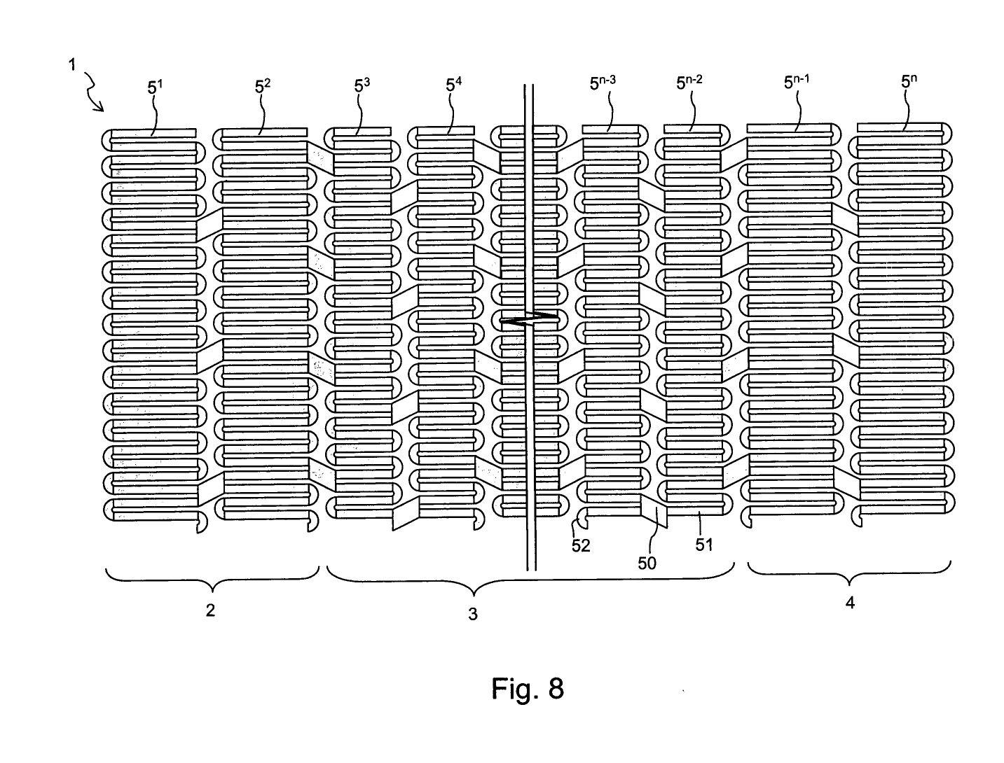

FIG. 8 schematically represents the planar development of a stent according to the invention;

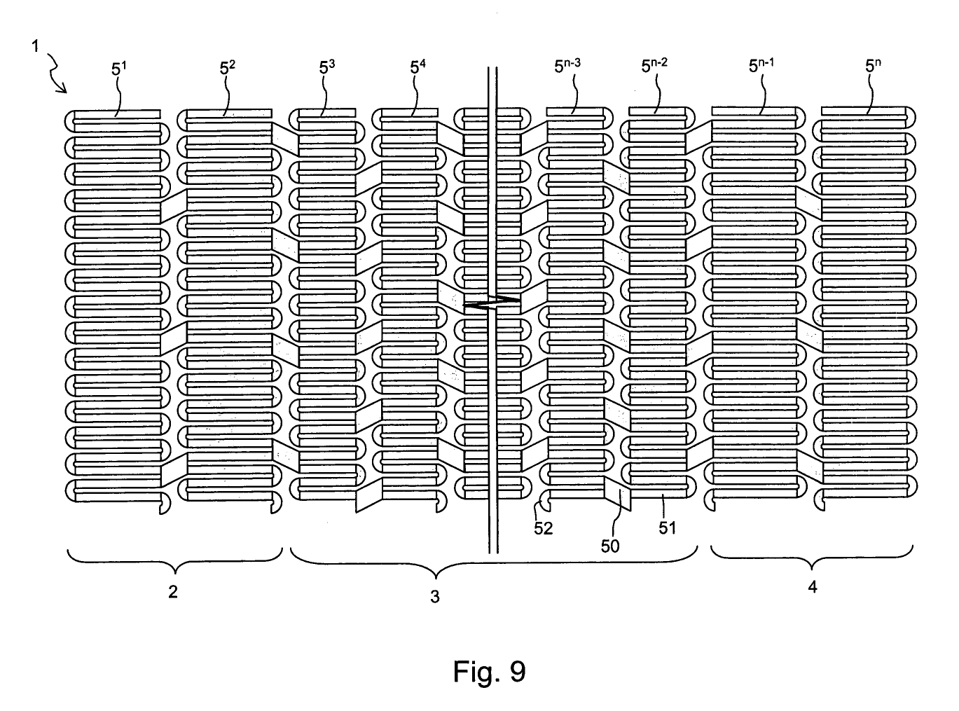

FIG. 9 schematically represents the planar development of a stent according to the invention.

With reference to the Figures, a stent according to the invention is indicated with 1, while the assembly of at least a first stent 1′ and a second stent 1″ is generally indicated with 10.

The stent 1 is of the type, broadly known per se, which is called Self-Expandable. That is, it is composed by a shape memory alloy (for example, Nitinol) which allows the stent to spontaneously adopt the expanded configuration, after any radial constraint has been removed.

The stent 1 extends along a longitudinal X-X axis. Therefore, each direction which is parallel to the X-X axis is called the axial direction. Herein below the left part of the Figures is conventionally considered as representing the proximal part of the stents and, vice versa, the right part of the drawings is conventionally considered as representing the distal part of the stents.

Each of the stents 1 according to the invention comprises a proximal section 2, a central section 3, and a distal section 4. The proximal section 2 and distal section 4 provide a radial force which is essentially equal to half the radial force being provided by the central section 3. Thereby, by overlapping the distal section 4″ of the second stent 1″ to the proximal section 2′ of the first stent 1′, the radial force which is provided by the overlapped sections is nearly equal to the radial force which is provided by the central sections 3′, 3″ of the two stents 1′, 1″.

In FIG. 6, a diagram is provided of the average radial forces exerted by the different sections of the stent 1 according to the invention The first and third columns of the histogram (marked as End) represent the average radial force in percent exerted by the proximal section 2 and the distal section 4 relative to the average radial force exerted by the central section 3 (second column, marked as Middle).

In accordance with an embodiment, the stent 1 comprises a plurality of serpentines 5. Each serpentine 5 comprises a plurality of struts 51 which are jointed one to the other by a plurality of bends 52.

Herein below it is assumed that the stent has a general number n of serpentines 5. Conventionally, the serpentines 5 will be identified by an apex which indicates the progressive position starting from the proximal end to the distal end.

Each serpentine is connected to at least one serpentine adjacent thereto by means of links 50. The proximal end serpentine 5¹ and the distal end serpentine 5ⁿ are connected by means of links 50 to a single adjacent serpentine (5² and 5ⁿ⁻¹, respectively), while each of the other serpentines 5′ is connected, by means of links 50, to the two adjacent serpentines 5^{x-1} and 5^{x+1}.

In accordance with an embodiment, the stent 1 according to the invention comprises serpentines 5 with struts having different lengths along the X-X axis. In particular, in the proximal section 2 and distal section 4, the struts 51 of the serpentines 5 are longer than the struts 51 of the serpentines 5 in the central section 3 of the same stent 1. Such characteristic is schematized in the FIGS. 7, 8, and 9, and it is illustrated in FIG. 4.

As it will be noted, in the embodiment of FIG. 4, the proximal section 2 of the stent 1 comprises three serpentines 5. The proximal end serpentine 5¹ has a total axial extension (given by the axial length of the struts 51 summed to the axial dimension of the bends 52) of 3.55 mm. The subsequent two serpentines 5² and 5³ of the proximal section 2 both have a whole axial extension of 3 mm. In a perfectly symmetric manner, the distal section 4 also comprises three serpentines 5. The distal end serpentine 5¹⁹ has a whole axial extension of 3.55 mm. The preceding two serpentines 5¹⁸ and 5¹⁷ of the distal section 4 both have a whole axial extension of 3 mm. All the serpentines 5⁴⁺¹⁶ of the central section 3 have a whole axial extension of 2.454 mm.

The higher length of the struts 51 of the serpentines 5 contributes to decrease the radial force F exerted by the proximal 2 and distal 4 sections.

In accordance with an embodiment, the stent 1 according to the invention comprises serpentines 5 that are connected by a different number of links 50 along the X-X axis. In particular, in the proximal 2 and distal 4 sections, the number of links 50 is less than that of the links 50 which are present in the central section 3 of the same stent 1. Such characteristic is outlined in the FIGS. 8 and 9.

In FIG. 8, it can be seen that the proximal end serpentine 5¹ is connected to the subsequent serpentine 5² by means of three links 50, and that the serpentine 5² is connected to the subsequent serpentine 5³ by means of four links 50. All the serpentines 5^{X} of the central section 3 are connected by means of four links 50, while the serpentine 5ⁿ⁻¹ is connected to the subsequent distal end serpentine 5ⁿ by means of three links 50.

In FIG. 9, it can be seen that the proximal end serpentine 5¹ is connected to the subsequent serpentine 5² by means of three links 50; that the serpentine 5² is connected to the subsequent serpentine 5³ by means of four links 50; and that the serpentine 5³ is connected to the subsequent serpentine 5⁴ by means of five links 50. All the serpentines 5^{X} of the central section 3 are connected by means of five links 50. The serpentine 5ⁿ⁻³ is connected to the subsequent serpentine 5ⁿ⁻² by means of five links 50; the serpentine 5ⁿ⁻² is connected to the subsequent serpentine 5ⁿ⁻¹ by means of four links 50; the serpentine 5ⁿ⁻¹ is connected to the subsequent distal end serpentine 5ⁿ by means of three links 50.

The lesser number of links 50 contributes to decrease the radial force F exerted by the proximal 2 and distal 4 sections.

In accordance with some embodiments, the proximal 2 and distal 4 sections of the stent 1 have a radial thickness which is lower than that of the central section 3. The lower radial thickness contributes to decrease the radial force F exerted by the proximal 2 and distal 4 sections.

In accordance with some embodiments, the stent 1 comprises, in a manner known per se, markers 6 made in a radiopaque material (for example, Tantalum, Gold, Platinum, or Tungsten). In fact, the shape memory alloys such as Nitinol are nearly transparent to radioscopy, therefore the radiopaque markers 6 increase the stent 1 visibility during the radioscopy-controlled operation.

In accordance with some embodiments, for example that in FIG. 4, the stent 1 comprises (with an apex convention analogue to that which has been employed above in order to identify the serpentines):
a first marker 6¹ at the proximal end of the stent 1; and
a second marker 6² at the distal end of the stent 1.

In accordance with other embodiments, for example that in FIG. 5, the stent 1 comprises:
a first marker 6¹ at the proximal end of the stent 1;
a second marker 6² at the distal end of the proximal section 2;
a third marker 6³ at the proximal end of the distal section 4; and
a fourth marker 6⁴ at the distal end of the stent 1.

In accordance with an embodiment, for example that in FIG. 4, the radiopaque markers 6 have a greater size than those which are typically employed. In particular, the marker 6 has an axial extension which is above 50%, preferably above 65%, still more preferably above 70%, the whole axial extension of the serpentine 5 which it is housed in.

For example, in the embodiment of FIG. 5, the marker 6 has an axial extension of 2.5 mm, as compared with 3.55 mm axial extension of the serpentine 5 in which it is housed. Therefore, the marker 6 has an axial extension above 70% the whole axial extension of the serpentine 5 which it is housed in.

The configuration of the stent 1 represented in FIG. 5 allows, during the surgery, radioscopically controlling, in an extremely accurate manner, the overlapping of the distal section 4″ of the second stent 1″ to the proximal section 2′ of the previously implanted first stent 1′. Such overlapping is completed when the axial positions marked by the markers 6⁴ and 6³ of the second stent 1″ correspond, respectively, to the axial positions marked by the markers 6² and 6¹ of the previously implanted first stent 1′.

The method for using the assembly 10 according to the invention comprises the steps of:
providing a first stent 1′ in a collapsed configuration;
introducing the first stent 1′ along the vessel of a patient affected by a lesion to the distal end of the lesion;
bringing the first stent 1′ from the collapsed configuration to the expanded configuration;
providing a second stent 1″ in a collapsed configuration;
introducing the second stent 1″ along the same vessel to the lesion;
introducing the distal portion 4″ of the second stent 1″ inside the proximal portion 2′ of the first stent 1′;
bringing the second stent 1″ from the collapsed configuration to the expanded configuration.

The method for using the assembly 10 can provide for other steps of arrangement, introduction, and expansion, of subsequent stents 1, in accordance with a modular logic, until reaching the proximal end of the lesion.

In accordance with some embodiments of the method, the step of introducing the distal portion 4″ of the second stent 1″ within the proximal portion 2′ of the first stent 1′ can by advantageously carried out by radioscopically controlling the relative position of the two stents 1″ and 1′ through the radiopaque markers 6.

As those skilled in the art should appreciate from what has been set forth above, the use of an assembly 10 according to the invention allows the treatment of diseases of an a priori indefinite extension. To the implant of a first stent 1, an indefinite number of other stents can a priori follow, without ever overcoming the desired radial force, in any length of the vessel.

It should be appreciated that only some particular embodiments of the stent being the object of the present invention have been described, to which those skilled in the art will be able to carry out all the modifications required for adapting the same to particular applications, without departing from the scope of protection of the present invention.

1. An assembly (10) comprising at least a first stent (1′), and a second stent (1″), each stent (1) comprising a proximal section (2), a central section (3), and a distal section (4), wherein the proximal section (2) and distal section (4) provide a radial force which is essentially equal to half the radial force being provided by the central section (3), such that, by overlapping the distal section (4″) of the second stent (1″) to the proximal section (2′) of the first stent (1′), the radial force being provided by the overlapped sections is nearly equal to the radial force which is provided by the central sections (3′, 3″) of the two stents (1′, 1″).

2. The assembly (10) according to claim 1, wherein the stent (1) comprises a plurality of serpentines (5), each serpentine comprising a plurality of struts (51) being joined to each other by a plurality of bends (52), and being connected to at least one serpentine adjacent thereto by means of links (50).

3. The assembly (10) according to claim 2, wherein the serpentines (5) of the proximal (2) and distal (4) sections have struts (51) which are longer than the struts (51) of the serpentines (5) of the central section (3) of the stent (1).

4. The assembly (10) according to claim 2, wherein the serpentines (5) of the proximal (2) and distal (4) sections are connected by a number of links (50) which is lower than the number of the links (50) which connect the serpentines (5) of the central section (3) of the same stent (1).

5. The assembly (10) according to claim 1, wherein the proximal (2) and distal (4) sections of the stent (1) have a radial thickness which is lower than the central section (3).

6. The assembly (10) according to claim 1, wherein the stent (1) comprises at least one marker (6) that is made of a radiopaque material being selected from the group consisting of Tantalum, Gold, Platinum, and Tungsten.

7. The assembly (10) according to claim 6, wherein the stent (1) comprises:
a first marker (6¹) at the proximal end of the stent (1); and
a second marker (6²) at the distal end of the stent (1).

8. The assembly (10) according to claim 6, wherein the stent (1) comprises:
a first marker (6¹) at the proximal end of the stent (1);
a second marker (6²) at the distal end of the proximal section (2);
a third marker (6³) at the proximal end of the distal section (4); and
a fourth marker (6⁴) at the distal end of the stent (1).

9. The assembly (10) in accordance with claim 6, wherein the at least one radiopaque marker (6) has an axial extension above 50%, preferably above 65%, still more preferably above 70%, of the total axial extension of the serpentine (5) in which it is housed.

10. A method for using an assembly (10) according to claim 1, comprising the steps of:
providing the first stent (1′) in a collapsed configuration;
introducing the first stent (1′) along the vessel of a patient affected by a lesion to the distal end of the lesion;
bringing the first stent (1′) from the collapsed configuration to the expanded configuration;
providing the second stent (1″) in a collapsed configuration;
introducing the second stent (1″) along the same vessel to the lesion;
introducing the distal portion (4″) of the second stent (1″) inside the proximal portion (2′) of the first stent (1′);
bringing the second stent (1″) from the collapsed configuration to the expanded configuration.

11. A stent (1) comprising a proximal section (2), a central section (3), and a distal section (4), wherein the proximal (2) and distal (4) sections provide a radial force which is essentially equal to a half of the radial force which is provided by the central section (3), such that, by overlapping the distal section (4″) of a second stent (1″) to the proximal section (2′) of a first stent (1′), the radial force which is provided by the overlapped sections is nearly equal to the radial force which is provided by the central sections (3′, 3″) of the two stents (1′, 1″).
